(19) United States
(12) Patent Application Publication
(10) Pub. No.: US 2023/0023463 A1
(43) Pub. Date: Jan. 26, 2023

(54) COMPOSITION FOR PROMOTING HAIR THICKENING, COMPOSITION FOR SUSTAINING GROWTH PHASE IN HAIR CYCLE AND COMPOSITION FOR PROMOTING VEGF PRODUCTION, COMPOSITION FOR PROMOTING TRANSITION FROM RESTING PHASE TO GROWTH PHASE IN HAIR CYCLE AND COMPOSITION FOR PROMOTING FGF7 PRODUCTION, AND COMPOSITION FOR PROMOTING HAIR CYCLE NORMALIZATION

(71) Applicant: AJINOMOTO CO., INC., Tokyo (JP)

(72) Inventors: Yukino Katayama, Kanagawa (JP); Kana Suzuki, Kanagawa (JP); Katsuya Suzuki, Kanagawa (JP); Chie Tanaka, Kanagawa (JP); Yoshinobu Takino, Kanagawa (JP); Yasuyo Suga, Kanagawa (JP); Reiko Ichikawa, Kanagawa (JP); Shinobu Akiyoshi, Kanagawa (JP)

(73) Assignee: AJINOMOTO CO., INC., Tokyo (JP)

(21) Appl. No.: 17/950,387

(22) Filed: Sep. 22, 2022

Related U.S. Application Data
(63) Continuation of application No. PCT/JP2021/012524, filed on Mar. 25, 2021.

(30) Foreign Application Priority Data
Mar. 26, 2020 (JP) 2020-056850

(51) Int. Cl. A61K 8/44; A61K 8/49; A61Q 7/00

(52) U.S. Cl. CPC A61K 8/44; A61K 8/492; A61Q 7/00

(57) ABSTRACT

According to the present invention, a composition that has high safety, can be continuously used for a long period, promotes production of VEGF and FGF7 involved in hair growth, and affords good prophylactic or improving effect on thinning hair or hair loss can be provided. The present invention relates to a composition for promoting hair thickening, containing one or more members selected from the group consisting of citrulline, tryptophan, glutamine, and ornithine, a composition for maintaining the anagen phase in the hair cycle and a composition for promoting VEGF production, each containing one or more members selected from the group consisting of citrulline, tryptophan, and glutamine, a composition for promoting transition from the telogen phase to the anagen phase in the hair cycle and a composition for promoting FGF7 production, each containing ornithine, and a composition for promoting normalization of the hair cycle, containing one or more members selected from the group consisting of citrulline, tryptophan, glutamine, and ornithine.

This application is a Continuation of, and claims priority under 35 U.S.C. § 120 to, International Application No. PCT/JP2021/012524, filed Mar. 25, 2021, and claims priority therethrough under 35 U.S.C. § 119 to Japanese Patent Application No. 2020-056850, filed Mar. 26, 2020, the entireties of which, as well as all citations cited herein, are incorporated by reference herein.

### BACKGROUND

### Technical Field

The present invention relates to a composition for promoting hair thickening. In addition, the present invention relates to a composition for maintaining the anagen phase of the hair cycle, a composition for promoting VEGF production, a composition for promoting transition from the telogen phase to the anagen phase of the hair cycle, a composition for promoting FGF7 production, and a composition for promoting normalization of the hair cycle.

### Background Art

Many people have long been troubled by thinning hair and hair loss. Hair loss, or alopecia, is frequently observed and results from various causes, such as androgenetic alopecia caused by the action of androgen and female pattern alopecia caused by aging and reduction of female hormone.

The hair growth cycle includes the following phases: the anagen phase in which hair matrix cells actively differentiate and proliferate after new hair growth until the growth stops, the catagen phase which is the process of transition to the telogen phase after the end of the anagen phase, and the telogen phase during which the hair root degenerates to reach hair loss and thereafter hair growth begins anew is known. Abnormality of this hair cycle is known to be one of the causes of alopecia.

Specifically, it has been reported that female pattern alopecia develops because transition from the telogen phase to the anagen phase of the hair cycle does not occur, and the new hair growth is suppressed, causing thinning hair, and that androgenetic alopecia develops because the anagen phase is shortened in the hair cycle, resulting in thin short hair.

Agents for hair growth and agents for new hair growth, via treating or improving androgenetic alopecia and female pattern alopecia containing various active ingredients are currently on the market.

Active ingredients for external use include carpronium chloride having a blood circulation promoting action, trans-3,4′-dimethyl-3-hydroxyflavanone (t-flavanone) having a proliferation promoting action on hair matrix cells, and adenosine, cytopurine (6-benzylaminopurine), pentadecanoic acid glyceride, and the like having a new hair growth-promoting action. Particularly, the effectiveness of minoxidil which is an antihypertensive agent has been reported.

Oral medicines include finasteride and dutasteride, which are testosterone 5-α reductase inhibitors, and are effective for androgenetic alopecia.

However, the improving effects of the above-mentioned components, which are externally applied in anticipation of blood circulation promoting action, hair matrix cell proliferation promoting action, new-hair-growth promoting action, and the like on hair loss and thinning hair are not sufficient. Minoxidil has been reported to cause skin symptoms such as pruritus, erythema, desquamation, hair follicle inflammation, contact dermatitis, and the like.

In addition, finasteride and dutasteride cannot be expected to be effective for female pattern alopecia since androgen is not involved.

The ability of amino acids which are components derived from living organisms, to promote hair growth or new hair growth, have also been examined, and it has been reported that metabolic intermediates of the urea cycle such as arginine, ornithine, citrulline, argininosuccinic acid, and the like and derivatives thereof can be preferably used in external compositions for increasing, enhancing, or maintaining hair growth (Patent Literature 1).

In addition, a hair treatment composition containing a substance that promotes new hair growth containing a protein biosynthesis stimulation substance which includes soybean peptide, amino acid, glutamine, glutamic acid, and protein hydrolysate extract, and an antidandruff agent is disclosed (Patent Literature 2).

However, with regard to citrulline and ornithine, which are intermediates of the urea cycle, Patent Literature 1 only shows the observation results of hair growth (hair length increment) in isolated and cultured hair follicles.

In addition, Patent Literature 2 does not specifically indicate the new-hair-growth promoting effect of amino acids themselves, and their effectiveness is unknown.

To promote healthy hair growth and improve thinning hair and hair loss, it is considered effective not only to promote hair length increment and new hair growth, but also to normalize the hair cycle and to thicken hair, that is, to promote the thickening of hair.

### CITATION LIST

### Patent Literature

PTL 1: Japanese Translation of PCT Application Publication No. h8-502509
PTL 2: Japanese Translation of PCT Application Publication No. 2011-500760

### SUMMARY

Therefore, it is described herein that Vascular endothelial growth factor (VEGF) and fibroblast growth factor 7 (FGF7), which have been reported in recent years as growth factors involved in hair growth, can be included in a composition that promotes the production of these factors, promotes the thickening of hair, normalizes the hair cycle, and affords a good preventive or improving effect on thinning hair and hair loss.

It is described herein that each of tryptophan, glutamine, and citrulline can promote VEGF production in hair papilla cells, promote division and proliferation of hair matrix cells, suppress shortening of the anagen phase of hair, normalize the hair cycle by maintaining the anagen phase of hair, and promote thickening of hair by promoting proliferation of hair papilla cells.

In addition, ornithine has been found to have the ability to promote the FGF7 production in hair papilla cells, shorten the telogen phase which is from the degeneration of the hair root until a hair begins to grow anew, promote the transition from the telogen phase to the anagen phase to normalize the hair cycle, and promote the division and proliferation of hair matrix cells, thus promoting the new hair growth, and further that ornithine can promote proliferation of hair papilla cells to promote hair thickening.

Furthermore, by formulating a composition for promoting hair thickening, a composition for maintaining the anagen phase of the hair cycle, a composition for promoting VEGF production, a composition for promoting transition from the telogen phase to the anagen phase of the hair cycle, a composition for promoting FGF7 production, and a composition for promoting normalization of the hair cycle, each containing the above-mentioned amino acid, the above-mentioned problems have been solved.

That is, the present invention is described as follows:

It is an aspect of the present invention to provide a composition for promoting hair thickening, comprising one or more members selected from the group consisting of citrulline, tryptophan, glutamine, and ornithine.

It is a further aspect of the present invention to provide the composition as described above, wherein a total content of one or more members selected from the group consisting of citrulline, tryptophan, glutamine, and ornithine is 0.1 μM to 1 M.

It is a further aspect of the present invention to provide the composition as described above, wherein the composition comprises one or more members selected from the group consisting of citrulline, tryptophan, and glutamine, and ornithine.

It is a further aspect of the present invention to provide the composition as described above, wherein a content ratio of one or more members selected from the group consisting of citrulline, tryptophan, and glutamine, and ornithine (one or more members selected from the group consisting of citrulline, tryptophan, and glutamine:ornithine) is 1:0.1 to 1:0.5 in molar ratio.

It is a further aspect of the present invention to provide a composition for maintaining the anagen phase of the hair cycle, comprising one or more members selected from the group consisting of citrulline, tryptophan, and glutamine.

It is a further aspect of the present invention to provide the composition as described above, wherein a total content of one or more members selected from the group consisting of citrulline, tryptophan, and glutamine is 0.1 μM to 1 M.

It is a further aspect of the present invention to provide a composition for promoting VEGF production, comprising one or more members selected from the group consisting of citrulline, tryptophan, and glutamine.

It is a further aspect of the present invention to provide the composition as described above, wherein a total content of one or more members selected from the group consisting of citrulline, tryptophan, and glutamine is 0.1 μM to 1 M.

It is a further aspect of the invention present to provide a composition for promoting transition from the telogen phase to the anagen phase of the hair cycle, comprising ornithine.

It is a further aspect of the present invention to provide the composition as described above, wherein a content of the ornithine is 1 μM to 1 M.

It is a further aspect of the present invention to provide a composition for promoting FGF7 production, comprising ornithine.

It is a further aspect of the present invention to provide the composition as described above, wherein a content of the ornithine is 1 μM to 1 M.

It is a further aspect of the present invention to provide a composition for promoting normalization of the hair cycle, comprising one or more members selected from the group consisting of citrulline, tryptophan, glutamine, and ornithine.

It is a further aspect of the present invention to provide the composition as described above, wherein a total content of one or more members selected from the group consisting of citrulline, tryptophan, glutamine, and ornithine is 0.1 μM to 1 M.

It is a further aspect of the present invention to provide the composition as described above, wherein the composition comprises one or more members selected from the group consisting of citrulline, tryptophan, and glutamine, and ornithine.

It is a further aspect of the present invention to provide the composition as described above, wherein a content ratio of one or more members selected from the group consisting of citrulline, tryptophan, and glutamine, and ornithine (one or more members selected from the group consisting of citrulline, tryptophan, and glutamine:ornithine) is 1:0.1 to 1:0.5 in molar ratio.

It is a further aspect of the present invention to provide a food for preventing or improving thinning hair or hair loss, comprising either or both of the composition for promoting VEGF production as described above and the composition for promoting FGF7 production as described above.

It is a further aspect of the present invention to provide a topical composition for preventing or improving thinning hair or hair loss, comprising either or both of the composition for promoting VEGF production as described above and the composition for promoting FGF7 production as described above.

It is a further aspect of the present invention to provide the topical composition as described above, wherein the composition is a topical quasi-drug for the skin.

It is a further aspect of the present invention to provide the topical composition as described above, wherein the composition is a hair cosmetic or a scalp cosmetic.

It is a further aspect of the present invention to provide a composition for preventing or improving androgenetic alopecia, comprising the composition for maintaining the anagen phase of the hair cycle as described above.

It is a further aspect of the present invention to provide a composition for preventing or improving androgenetic alopecia, comprising the composition for promoting VEGF production as described above.

It is a further aspect of the present invention to provide a composition for preventing or improving female pattern alopecia, comprising the composition for promoting transition from the telogen phase to the anagen phase of the hair cycle as described above.

It is a further aspect of the present invention to provide a composition for preventing or improving female pattern alopecia, comprising the composition for promoting FGF7 production as described above.

It is a further aspect of the present invention to provide a composition as described above, wherein the composition suppresses or ameliorates scalp itchiness.

A composition for promoting hair thickening is provided.

The composition for promoting hair thickening can promote production of VEGF or FGF7 in hair papilla cells, and proliferation of hair papilla cells, and grow long, thick, and healthy hair.

A composition for maintaining the anagen phase of the hair cycle is provided.

The composition for maintaining the anagen phase of the hair cycle can suppress shortening of the anagen phase of hair and maintain the anagen phase of hair, by promoting VEGF production in hair papilla cells.

A composition for promoting VEGF production in hair papilla cells is provided.

The composition for promoting VEGF production can promote VEGF production in hair papilla cells, promote division and proliferation of hair matrix cells, suppress shortening of the anagen phase of hair, normalize the hair cycle by maintaining the anagen phase of hair, and promote thickening of hair by promoting proliferation of hair papilla cells.

Therefore, the composition for maintaining the anagen phase of the hair cycle and the composition for promoting VEGF production can be utilized, particularly as a hair-growing agent.

A composition for promoting transition from the telogen phase to the anagen phase of the hair cycle is provided.

The composition for promoting transition from the telogen phase to the anagen phase of the hair cycle can shorten the telogen phase which is from the degeneration of the hair root until a hair begins to grow anew, and promote the transition from the telogen phase to the anagen phase of hair, by promoting FGF7 production in hair papilla cells.

A composition for promoting FGF7 production in hair papilla cells is provided.

The composition for promoting FGF7 production can shorten the telogen phase which is from the degeneration of the hair root until a hair begins to grow anew by promoting FGF7 production in hair papilla cells, normalize the hair cycle by promoting the transition from the telogen phase to the anagen phase, and promote growing of a new hair by promoting the division and proliferation of hair matrix cells.

Therefore, the composition for promoting transition from the telogen phase to the anagen phase of the hair cycle and the composition for promoting FGF7 production are used, particularly as a new-hair-growing agent.

A composition for promoting normalization of the hair cycle is provided.

The composition for promoting the normalization of the hair cycle can normalize the hair cycle by either or both of maintaining the anagen phase of the hair cycle by promoting VEGF production, and of promoting transition from the telogen phase to the anagen phase of the hair cycle by promoting FGF7 production.

The composition for promoting hair thickening, the composition for maintaining the anagen phase of the hair cycle, and the composition for promoting VEGF production, the composition for promoting transition from the telogen phase to the anagen phase of the hair cycle and the composition for promoting FGF7 production, and the composition for promoting normalization of the hair cycle can favorably improve or prevent thinning hair or hair loss, and particularly useful for androgenetic alopecia and female pattern alopecia.

Furthermore, the composition for promoting hair thickening, the composition for maintaining the anagen phase of the hair cycle, and the composition for promoting VEGF production, the composition for promoting transition from the telogen phase to the anagen phase of the hair cycle and the composition for promoting FGF7 production, and the composition for promoting normalization of the hair cycle contain amino acids that have been frequently formulated as active ingredients in compositions, are highly safe, can be used not only for external use, but also for oral ingestion or oral administration, and can be used continuously for a long period of time.

### BRIEF DESCRIPTION OF DRAWINGS

FIG. 1 is a diagram showing the influence of citrulline on VEGF production in hair papilla cells in Experimental Example 1. In the Figure, “**” indicates a significant difference at P<0.01 from the control, and “***” indicates a significant difference at P<0.001 from the control.

FIG. 2 is a diagram showing the influence of tryptophan on VEGF production in hair papilla cells in Experimental Example 1. In the Figure, “**” indicates a significant difference at P<0.01 from the control, and “***” indicates a significant difference at P<0.001 from the control.

FIG. 3 is a diagram showing the influence of glutamine on VEGF production in hair papilla cells in Experimental Example 1. In the Figure, “**” indicates a significant difference at P<0.01 from the control, and “***” indicates a significant difference at P<0.001 from the control.

FIG. 4 is a diagram showing the influence of ornithine on FGF7 production in hair papilla cells in Experimental Example 2. In the Figure, “*” indicates a significant difference at P<0.05 from the control, and “*” indicates a significant difference at P<0.01 from the control.

FIG. 5 is a diagram showing the influence of citrulline on proliferation of hair papilla cells in Experimental Example 3. In the Figure, “**” indicates a significant difference at P<0.01 from the control.

FIG. 6 is a diagram showing the influence of ornithine on proliferation of hair papilla cells in Experimental Example 4. In the Figure, “*” indicates a significant difference at P<0.05 from the control, and “**” indicates a significant difference at P<0.01 from the control.

FIG. 7 is a diagram showing the amount of change in score for each evaluation item regarding hair in Experimental Example 5 before and after ingestion of the test food. In the Figure, “*” indicates a significant difference at P<0.05 from before ingestion (pre), “**” indicates a significant difference at P<0.01 from before ingestion (pre), and “***” indicates a significant difference at P<0.001 from ingestion (pre).

FIG. 8 is a diagram showing the relationship between the content ratio of citrulline and ornithine hydrochloride, and the plasma concentration of citrulline in Experimental Example 6. In the Figure, A shows the results 30 min after administration of the test solution, B shows the results 1 hr after administration of the test solution, and C shows the results 2 hr after administration of the test solution. In the Figure, “DW” indicates a distilled water administration group. Each “*” indicates a significant difference at P<0.05, each “**” indicates a significant difference at P<0.01, and each “***” indicates a significant difference at P<0.001.

FIG. 9 is a diagram showing the relationship between the content ratio of citrulline and ornithine hydrochloride, and the plasma concentration of ornithine in Experimental Example 6. In the Figure, A shows results 30 min after administration of the test solution, B shows results 1 hr after administration of the test solution, and C shows results 2 hr after administration of the test solution. In the Figure, “DW” indicates a distilled water administration group. Each “*” indicates a significant difference at P<0.05, each “**” indicates a significant difference at P<0.01, and each “***” indicates a significant difference at P<0.001.

### DETAILED DESCRIPTION OF THE EXEMPLARY EMBODIMENTS

A composition for promoting hair thickening is provided herein.

The composition for promoting hair thickening (hereinafter sometimes to be referred to as “the thickening promoting composition”) contains one or more member selected from the group consisting of citrulline, tryptophan, glutamine, and ornithine.

Citrulline, tryptophan, and glutamine each have actions of promoting proliferation of hair papilla cells and promoting hair thickening, via promotion of VEGF (vascular endothelium growth factor) production in hair papilla cells.

In addition, ornithine has actions of promoting FGF7 (fibroblast growth factor 7) production in hair papilla cells and proliferation of hair papilla cells, and promoting hair thickening.

Citrulline has the general chemical name of 2-amino-5-(carbamoylamino)pentanoic acid, and is an amino acid of the urea cycle.

Tryptophan has the general chemical name of 2-amino-3-(indolyl)propionic acid, and is one of the aromatic amino acids.

Glutamine has the general chemical name of 2-amino-4-carbamoylbutanoic acid, and is an amino acid having an amide on the side chain.

Ornithine has the general chemical name of 2,5-diamino pentanoic acid, and is an amino acid of the urea cycle.

The citrulline, tryptophan, glutamine, and ornithine may be any of L-form, D-form and DL-form. The L-form and DL-form are preferably used, and L-form is more preferably used.

In addition, the “citrulline”, “tryptophan”, “glutamine”, and “ornithine” may be not only a free form but also in a salt form. The terms “citrulline”, “tryptophan”, “glutamine”, and “ornithine” are each a concept encompassing even a salt. The salt form is not particularly limited as long as it is a pharmacologically acceptable salt, and acid addition salt, salt with base and the like can be encompassed.

Specifically, salts with inorganic bases, salts with organic bases, salts with inorganic acids, salts with organic acids, salts with amino acid and the like can be encompassed.

Examples of the salts with inorganic bases include salts with alkali metals such as lithium, sodium, potassium and the like, salts with alkaline earth metals such as magnesium, calcium and the like, ammonium salt and the like.

Examples of the salts with organic bases include salts with alkanolamine such as monoethanolamine, diethanolamine, triethanolamine and the like, salts with heterocyclic amine such as morpholine, piperidine and the like, and the like.

Examples of the salts with inorganic acids include salts with hydrohalic acid, such as hydrochloric acid, hydrobromic acid, hydroiodic acid, etc.; sulfuric acid, nitric acid, phosphoric acid and the like, and the like.

Examples of the salts with organic acids include salts with monocarboxylic acid such as formic acid, acetic acid, propanoic acid and the like; salts with saturated dicarboxylic acid such as oxalic acid, malonic acid, malic acid, succinic acid and the like; salts with unsaturated dicarboxylic acid such as maleic acid, fumaric acid and the like; salts with tricarboxylic acid such as citric acid and the like; salts with keto acid such as α-ketoglutaric acid and the like, and the like.

Examples of the salts with amino acid include salts with aliphatic amino acid such as alanine and the like; salts with aromatic amino acid such as tyrosine and the like; salts with basic amino acid such as arginine and the like; salts with acidic amino acid such as aspartic acid, glutamic acid and the like; salts with amino acid forming lactam such as pyroglutamic acid and the like; and the like.

The above-mentioned salts may each be a hydrate (hydrate salt), and examples of the hydrate include 1 hydrate to 6 hydrate and the like.

“Citrulline”, “tryptophan”, “glutamine”, and “ornithine” in the above-described free form or a salt form may be used singly, or two or more thereof may be used in combination.

Each of “citrulline”, “tryptophan”, “glutamine”, and “ornithine” in a free form, hydrochloride thereof, and the like can be used preferably in the method as described herein.

“Citrulline”, “tryptophan”, “glutamine”, and “ornithine” in a free form or in the form of a salt may be extracted from animals, plants or the like, which are naturally present, and purified, or obtained by a chemical synthesis method, a fermentation method, an enzyme method or a gene recombinant method. Commercially available products provided by each company may also be utilized.

In the thickening promoting composition, one member or two or more members selected from the group consisting of citrulline, tryptophan, glutamine, and ornithine can be used.

When the thickening promoting composition is provided in a solution form, the total content of one or more members selected from the group consisting of citrulline, tryptophan, glutamine, and ornithine in the thickening promoting composition and the like is generally 0.1 μM to 1 M, preferably 0.3 μM to 500 mM, more preferably 1 μM to 100 mM.

Referring to the “total amount of these” or the “total amount of one or more members selected from the group consisting of citrulline, tryptophan, glutamine, and ornithine” or the “total amount of one or more members selected from the group consisting of citrulline, tryptophan, and glutamine”, when one selected from the group consisting of citrulline, tryptophan, glutamine, and ornithine, or the group consisting of citrulline, tryptophan, and glutamine is contained or used, it means the content or amount of the amino acid to be used; and when two or more are selected and contained or used in combination, it means the total contents or amount thereof.

Referring to the “total content”, when one is selected from the group consisting of citrulline, tryptophan, glutamine, and ornithine, or the group consisting of citrulline, tryptophan, and glutamine, and contained, it means the content of the amino acid, and when two or more are selected and contained, it means total amount of the contents thereof.

From the aspect of hair thickening promoting effect, the thickening promoting composition preferably contains one or more members selected from the group consisting of citrulline, tryptophan, and glutamine, and ornithine. A particular combination is citrulline and ornithine.

The content ratio of one or more members selected from the group consisting of citrulline, tryptophan, and glutamine, and ornithine, that is, one or more members selected from the group consisting of citrulline, tryptophan, and glutamine:ornithine, is preferably 1:0.1 to 1:0.5, more preferably 1:0.15 to 1:0.32, in molar ratio.

Provided herein is a composition for maintaining the anagen phase of the hair cycle based on the promotion of VEGF production. Further provided is a composition for promoting VEGF production.

VEGF (vascular endothelium growth factor) is a growth factor that is produced by hair papilla cells, promotes division and proliferation of hair matrix cells, is involved in maintaining the anagen phase of hair, and has an action of promoting proliferation of hair papilla cells.

The composition for maintaining the anagen phase of the hair cycle (hereinafter sometimes to be referred to as “the composition for maintaining the anagen phase”) and the composition for promoting VEGF production each contain one member or two or more members selected from the group consisting of citrulline, tryptophan, and glutamine.

Citrulline, tryptophan, and glutamine contained as active ingredients in each of the composition for maintaining the anagen phase and the composition for promoting VEGF production are as described above in the thickening promoting composition.

When the composition for maintaining the anagen phase and the composition for promoting VEGF production are provided in the form of solutions, the total content of one or more members selected from the group consisting of citrulline, tryptophan, and glutamine is generally 0.1 μM to 1 M, preferably 0.3 μM to 500 mM, more preferably 1 μM to 100 mM.

Also described herein is a composition for promoting transition from the telogen phase to the anagen phase of the hair cycle based on the promotion of VEGF production. Further described herein is a composition for promoting FGF7 production.

FGF7 (fibroblast growth factor 7) is a growth factor that is produced by hair papilla cells, is involved in the transition from the telogen phase to the anagen phase of hair, and has an action of promoting division and proliferation of hair matrix cells.

The composition for promoting transition from the telogen phase to the anagen phase of the hair cycle (hereinafter sometimes to be referred to as “the composition for promoting the anagen phase transition”) and the composition for promoting FGF7 production contain ornithine.

Ornithine is present as an active ingredient in each of the composition for promoting the anagen phase transition of the present invention and the composition for promoting FGF7 production as described above in the thickening promoting composition.

When the composition for promoting the anagen phase transition and the composition for promoting FGF7 production are provided in the form of solutions, the content of ornithine in each is generally 1 μM to 1 M, preferably 5 μM to 500 mM, more preferably 10 μM to 100 mM.

Furthermore, provided herein is a composition for promoting the normalization of the hair cycle that can normalize the hair cycle by either or both of the action of maintaining the anagen phase of the hair cycle by promoting VEGF production, and the action of promoting transition from the telogen phase to the anagen phase of the hair cycle by promoting FGF7 production.

The composition for promoting the normalization of the hair cycle contains one member or two or more members selected from the group consisting of citrulline, tryptophan, glutamine, and ornithine.

Citrulline, tryptophan, glutamine, and ornithine are present as active ingredients in the composition for promoting the normalization of the hair cycle as described above in the thickening promoting composition.

When the composition for promoting the normalization of the hair cycle is provided in the form of a solution, the total content of one or more members selected from the group consisting of citrulline, tryptophan, glutamine, and ornithine is generally 0.1 μM to 1 M, preferably 0.3 μN to 500 mM, more preferably 1 μM to 100 mM.

From the aspect of hair cycle normalization promoting effect, the composition for promoting the normalization of the hair cycle preferably contains one or more members selected from the group consisting of citrulline, tryptophan, and glutamine, and ornithine. A combination of citrulline and ornithine is a particular example.

The content ratio of one or more members selected from the group consisting of citrulline, tryptophan, and glutamine, and ornithine, that is, one or more members selected from the group consisting of citrulline, tryptophan, and glutamine:ornithine, is preferably 1:0.1 to 1:0.5, more preferably 1:0.15 to 1:0.32, in molar ratio.

The thickening promoting composition of the above-mentioned present invention, the composition for maintaining the anagen phase and the composition for promoting VEGF production, the composition for promoting the anagen phase transition and the composition for promoting FGF7 production, and the composition for promoting the normalization of the hair cycle can be respectively formulated as oral compositions in solid form, e.g., tablet, coated tablet, chewable tablet, pill, (micro)capsule, granule, fine granule, powder, and the like; liquid form, e.g., elixir, lemonade, syrup, suspension, emulsion, and the like; semi-solid form, e.g., jelly for oral administration and the like; and the like, topical preparations or topical quasi-drugs for the skin such as topical liquid, e.g., liniment, lotion, and the like; spray, e.g., external aerosol, pump spray, and the like; ointment, e.g., oleagenous ointment, water-soluble ointment, and the like; cream, e.g., oil-in-water type cream, water-in-oil type cream, and the like; gel, e.g., aqueous gel, oily gel, and the like; and the like, topical compositions such as cosmetics formulated for the hair or cosmetics formulated for the scalp, e.g., liquid, milky liquid, spray, paste, cream, gel, and the like, and the like.

Each composition as described herein can be prepared by formulation methods well known in the field of preparations, for example, the methods described in the Japanese Pharmacopoeia, seventeenth Edition, General Rules for preparation, [3] Monographs for Preparations.

In this case, various pharmacologically acceptable additives for preparations can be blended as necessary. The additive can be appropriately selected according to the form of each composition as described herein. For example, excipient, binder, disintegrant, lubricant, coating agent, base, solvent, diluent, solubilizing agent, solubilizer, emulsifier, dispersing agent, suspending agent, stabilizer, thickener, gelling agent, pH adjuster, antioxidant, antiseptic, preservative, corrigent, flavoring agent, sweetening agent, flavor, colorant, and the like can be encompassed.

Specifically, examples of the excipient include magnesium carbonate, titanium dioxide, saccharides such as lactose, etc., sugar alcohol such as mannitol, etc., casein, and the like.

Examples of the binder include gelatin, starch, cellulose and a derivative thereof, and the like.

Examples of the disintegrant include crospovidone, crystalline cellulose, and the like.

Examples of the lubricant include talc, magnesium stearate, and the like.

Examples of the coating agent include methylmethacrylate-butylmethacrylate-dimethylaminoethylmethacrylate copolymer, ethylacrylate-methylmethacrylate-trimethylammmonioethylmethacrylate chloride copolymer, and the like.

Examples of the base include animal oil, vegetable oil, hydrocarbon oil such as liquid paraffin, etc., polyethylene glycol, and the like.

Examples of the solvent include purified water, monovalent alcohol such as ethanol, etc., polyhydric alcohol such as glycerol, etc., and the like.

Examples of the emulsifier or dispersing agent include sorbitan fatty acid ester, glycerine fatty acid ester, polyoxyethylene sorbitan fatty acid ester, sucrose fatty acid ester, and the like.

Examples of the stabilizer include adipic acid, β-cyclodextrin, and the like.

Examples of the thickener include water-soluble polymer such as sodium polyacrylate, carboxyvinyl polymer, etc., polysaccharides such as sodium alginate, xanthan gum, tragacanth, etc., and the like.

Examples of the gelling agent include carboxyvinyl polymer, dextrin palmitate, and the like.

Examples of the pH adjuster include hydrochloric acid, sulfuric acid, acetic acid, citric acid, lactic acid, sodium hydroxide, potassium hydroxide, and the like.

Examples of the antioxidant include dibutylhydroxytoluene (BHT), butylhydroxyanisole (BHA), α-tocopherol, erythorbic acid, and the like.

Examples of the antiseptic or preservative include paraben such as methylparaben, etc., benzyl alcohol, sodium dehydroacetate, sorbic acid, and the like.

Examples of the corrigent or flavoring agent include ascorbic acid, erythritol, sodium L-glutamate, and the like.

Examples of the sweetening agent include aspartame, licorice extract, saccharin, and the like.

Examples of the flavor include 1-menthol, d-camphor, cineol, and the like.

Examples of the colorant include tar pigment (red No. 2, blue No. 1, yellow No. 4, etc.), inorganic pigment (red iron oxide, yellow iron oxide, black iron oxide, etc.), natural dye (annatto dye, turmeric dye, β-carotene, etc.), and the like.

The amount of use of each of the above-described compositions is appropriately determined according to the condition or degree of thinning hair or hair loss, gender, age, body weight of the subject to whom the composition is applied (hereinafter also to be referred to as the “application subject”), the form of the composition, use method, and the like.

As used herein, the “amount to be used” refers to an amount of ingestion or dose in the case of oral composition, and an amount to be applied in the case of external composition.

When the application subject is a human adult, the amount of use of each of the thickening promoting composition and the composition for promoting normalization of the hair cycle is generally 0.01 mg/kg body weight to 100 g/kg body weight, preferably 0.1 mg/kg body weight to 50 g/kg body weight, more preferably 1 mg/kg body weight to 10 g/kg body weight, per day, as a total amount of one or more members selected from the group consisting of citrulline, tryptophan, glutamine, and ornithine (when contained in a salt form, an amount converted to a free form), for ingestion or administration as an oral composition.

When applied externally or as a topical composition, the amount is generally 0.15 mg/cm² to 150 mg/cm², preferably 0.75 mg/cm² to 75 mg/cm², more preferably 1.5 mg/cm² to 15 mg/cm², per application.

When the application subject is a human adult, the amount of use of each of the composition for maintaining the anagen phase and the composition for promoting VEGF production is generally 0.01 mg/kg body weight to 100 g/kg body weight, preferably 0.1 mg/kg body weight to 50 g/kg body weight, more preferably 1 mg/kg body weight to 10 g/kg body weight, per day, as a total amount of one or more members selected from the group consisting of citrulline, tryptophan, and glutamine (when contained in a salt form, an amount converted to a free form), for ingestion or administration as an oral composition.

When applied externally or as a topical composition, the amount is generally 0.15 mg/cm² to 150 mg/cm², preferably 0.75 mg/cm² to 75 mg/cm², more preferably 1.5 mg/cm² to 15 mg/cm², per application.

When the application subject is a human adult, the amount of use of each of the composition for promoting the anagen phase transition and the composition for promoting FGF7 production is generally 0.01 mg/kg body weight to 100 g/kg body weight, preferably 0.1 mg/kg body weight to 50 g/kg body weight, more preferably 1 mg/kg body weight to 10 g/kg body weight, per day, as an amount of ornithine (when contained in a salt form, an amount converted to a free form), for ingestion or administration as an oral composition.

When applied externally or as a topical composition, the amount is generally 0.15 mg/cm² to 150 mg/cm², preferably 0.75 mg/cm² to 75 mg/cm², more preferably 1.5 mg/cm² to 15 mg/cm², per application.

The above-mentioned amount of ingestion or dose per day may be ingested or administered at one time, or in two or more divided portions (e.g., 2 to 5 portions).

The timing of use of each of the above-described compositions is not particularly limited. When ingested or administered as an oral composition, it may be ingested or administered before or after meal, or together with meal. When applied as an external composition, it can be appropriately used, for example, after washing of the face in the morning, after taking a bath, and the like.

While the frequency of applications or administrations of each of the above-described compositions is not particularly limited, since the expected hair thickening promoting effect and the like can be obtained, multiple times of continuous use by the application subject is preferred.

Since citrulline, tryptophan, glutamine, and ornithine are all amino acids that have been administered frequently and have high safety, each of the above-described compositions can be continuously used by the application subject. In order to effectively prevent or improve thinning hair or hair loss, use for a long period of time (e.g., 2 weeks or more) is preferred.

When each composition as described herein is provided as an oral composition, it can be formulated as a unit package form. The term “unit package form” means one or more units with a particular amount (e.g., intake per one time, etc.) that is/are packed in one container or package. For example, a unit package form with intake per one time as one unit is referred to as “unit package form for intake per one time”. A container or package used for the unit package form can be appropriately selected according to the form, and the like of each composition. For example, paper container or bag, plastic container or bag, pouch, aluminum can, steel can, glass bottle, pet bottle, PTP (press through pack) package sheet, and the like can be encompassed.

In addition, when each composition is provided in the form of an external or topical composition, the amount for single application can be discharged by a spray using an aerosol can or a pump container having a constant amount discharge mechanism.

The application subject includes mammals such as human, mouse, rat, hamster, rabbit, cat, dog, bovine, horse, donkey, swine, sheep, monkey, etc. When each composition is applied to an application subject animal other than human (hereinafter to be also simply referred to as “target animal”), each amount to be used thereof can be appropriately set according to the kind, sex, body weight, and the like of the target animal.

The composition for promoting hair thickening can promote production of VEGF or FGF7 in hair papilla cells and proliferation of hair papilla cells, and grow long, thick, and healthy hair.

The composition for maintaining the anagen phase and the composition for promoting VEGF production promote VEGF production in hair papilla cells and promote division and proliferation of hair matrix cells. They can correct the shortened anagen phase of hair, normalize the hair cycle by maintaining a normal period of the anagen phase, and promote hair length growth in those with thinning hair or hair loss.

The composition for promoting the anagen phase transition and the composition for promoting FGF7 production promote FGF7 production in hair papilla cells. They can normalize the hair cycle by promoting transition from the telogen phase to the anagen phase after degeneration of the hair root, and promote new hair growth by promoting the division and proliferation of hair matrix cells in those with thinning hair or hair loss.

Furthermore, the composition for promoting the normalization of the hair cycle can normalize the hair cycle by either or both of the action of maintaining the anagen phase of the hair cycle by promoting VEGF production, and the action of promoting transition from the telogen phase to the anagen phase of the hair cycle by promoting FGF7 production.

Therefore, each of the above-described compositions can improve thinning hair or hair loss in those with thinning hair or hair loss such as androgenetic alopecia, female pattern alopecia, and the like, and can prevent thinning hair or hair loss in middle-aged and elderly persons at a risk of development or progression of thinning hair or hair loss, and the like.

The composition for maintaining the anagen phase and the composition for promoting VEGF production are particularly effective for preventing or improving androgenetic alopecia.

In addition, the composition for promoting the anagen phase transition and the composition for promoting FGF7 production is particularly effective for preventing or improving female pattern alopecia.

Furthermore, each composition has an action of suppressing or ameliorating itchiness of the scalp, in addition to the above-mentioned hair growth-promoting, hair cycle-normalizing, and new-hair-growth promoting actions.

Hence, each composition can also be used as a composition for suppressing or ameliorating itchiness of the scalp.

The present invention also provides a hair-growing agent containing the thickening promoting composition, the composition for maintaining the anagen phase, or the composition for promoting VEGF production.

As used herein, the “hair growing” means to promote hair growth, and it means to lengthen hair and promote the proliferation of hair papilla cells to make the hair thick and elastic.

The hair-growing agent can be provided in the form of an oral preparation or a topical preparation according to the form of the thickening promoting composition and the like to be contained therein.

The content of the thickening promoting composition in the hair-growing agent is generally 0.000005 wt % to 99 wt %, preferably 0.00001 wt % to 95 wt %, more preferably 0.00005 wt % to 90 wt %, as a total content of one or more members selected from the group consisting of citrulline, tryptophan, glutamine, and ornithine (when contained in a salt form, calculated as an amount converted to a free form).

The content of the composition for maintaining the anagen phase or the composition for promoting VEGF production in the hair-growing agent is generally 0.000005 wt % to 99 wt %, preferably 0.00001 wt % to 95 wt %, more preferably 0.00005 wt % to 90 wt %, as a total content of one or more members selected from the group consisting of citrulline, tryptophan, and glutamine (when contained in a salt form, calculated as an amount converted to a free form).

The amount to be used, such as the amount to be ingested or the dose for oral preparation form, amount of application when used as a topical preparation form, of the hair-growing agent can be appropriately determined according to the condition and degree of thinning hair or hair loss of the subject for whom the hair-growing agent is used, the age, gender, and body weight of the subject, form of the hair-growing agent, and the like.

Specifically, the amount to be used of one or more members selected from the group consisting of citrulline, tryptophan, glutamine, and ornithine can be determined such that it is the amount of use per day as described above for the thickening promoting composition. In addition, the amount to be used of one or more members selected from the group consisting of citrulline, tryptophan, and glutamine can be determined such that it is the amount of use per day as described above for each of the composition for maintaining the anagen phase and the composition for promoting VEGF production.

The present invention further provides a new-hair-growing agent containing the composition for promoting the anagen phase transition or the composition for promoting FGF7 production.

As used herein, the “new-hair-growing” refers to the growth of hair from a state in which hair does not exist in hair follicles.

The new-hair-growing agent can be provided in the form of an oral preparation or a topical preparation according to the form of the composition for promoting the anagen phase transition and the like contained therein.

The content of the composition for promoting the anagen phase transition or the composition for promoting FGF7 production in the new-hair-growing agent is generally 0.0001 wt % to 90 wt %, preferably 0.0005 wt % to 85 wt %, more preferably 0.001 wt % to 80 wt %, as the content of ornithine (when contained in a salt form, calculated as an amount converted to a free form).

The amount to be used, such as the amount to be ingested or the dose in the case of oral preparation form, the amount of application when applied in topical preparation form, of the new-hair-growing agent can be appropriately determined according to the condition and degree of thinning hair or hair loss of the subject for whom the new-hair-growing agent is used, the age, gender, and body weight of the subject, form of the new-hair-growing agent, and the like. It can be determined such that the amount of use of ornithine is the amount of use per day mentioned above for the composition for promoting the anagen phase transition and the composition for promoting FGF7 production.

The hair-growing agent and the new-hair-growing agent may also be used in combination with other hair-growing components and new-hair-growing components. Specific examples of such hair-growing components and new-hair-growing components include blood circulation promoters such as carpronium chloride, swertianin, and the like; hair matrix cell stimulants such as trans-3,4′-dimethyl-3-hydroxyflavanone (t-flavanone), pantothenylethylether, and the like; new-hair-growth promoters such as adenosine, cytopurine (6-benzylaminopurine), pentadecanoic acid glycerides, and the like; and the like.

The hair-growing agent can be prepared by adding a general formulation additive to the thickening promoting composition, the composition for maintaining the anagen phase or the composition for promoting VEGF production as necessary, and according to a formulating means well known in the field of preparations, for example, the methods described in the Japanese Pharmacopoeia, seventeenth Edition, General Rules for preparation, [3] Monographs for Preparations and the like.

The new-hair-growing agent can be prepared by adding a general formulation additive to the composition for promoting the anagen phase transition or the composition for promoting FGF7 production as necessary, and in the same manner as described above.

The timing, frequency, and duration of administration/application, of the hair-growing agent and the new-hair-growing agent can be set in the same manner as the timing, frequency, and duration described above for the thickening promoting composition and the like.

The hair-growing agent and the new-hair-growing agent can be applied to those with thinning hair or hair loss such as androgenetic alopecia patients, female pattern alopecia patients, and the like, middle-aged and elderly persons at a risk of development or progression of thinning hair or hair loss, and the like. They can improve or prevent thinning hair or hair loss.

The present invention also provides a medicament for the prophylaxis or improvement of thinning hair or hair loss (hereinafter also to be referred to as “the medicament”).

The medicament contains either or both of the above-described composition for promoting VEGF production and the composition for promoting FGF7 production.

Either or both of the composition for promoting VEGF production and the composition for promoting FGF7 production can be directly used to give the medicament, or blended with the above-mentioned formulation additives such as excipient, solvent, diluent, and the like to give the medicament.

The content of the composition for promoting VEGF production in the medicament is generally 0.000005 wt % to 99 wt %, preferably 0.00001 wt % to 95 wt %, more preferably 0.00005 wt % to 90 wt %, as the total content of one or more members selected from the group consisting of citrulline, tryptophan, and glutamine (when contained in a salt form, calculated as an amount converted to a free form).

The content of the composition for promoting FGF7 production in the medicament is generally 0.0001 wt % to 90 wt %, preferably 0.0005 wt % to 85 wt %, more preferably 0.001 wt % to 80 wt %, as the content of ornithine (when contained in a salt form, as an amount converted to a free form).

From the aspects of the prophylactic or improving effect on thinning hair or hair loss, the medicament preferably contains both the composition for promoting VEGF production and the composition for promoting FGF7 production.

The composition for promoting VEGF production and the composition for promoting FGF7 production can be preferably contained in the medicament such that the content ratio of one or more members selected from the group consisting of citrulline, tryptophan, and glutamine, and ornithine (one or more members selected from the group consisting of citrulline, tryptophan, and glutamine:ornithine) is 1:0.1 to 1:0.5, more preferably 1:0.15 to 1:0.32.

The amount to be used, such as the dose in the case of oral preparation form, or the amount of application in the case of a topical preparation form, of the medicament can be appropriately determined according to the condition and degree of thinning hair or hair loss of the patients for whom the medicament is used, the age, gender, and body weight of the patients, medicament form, and the like.

Specifically, it can be determined such that the amount of use of one or more members selected from the group consisting of citrulline, tryptophan, and glutamine is the amount of use per day described above for the composition for maintaining the anagen phase and the composition for promoting VEGF production.

In addition, it can be determined such that the amount of use of ornithine is the amount of use per day described above for the composition for promoting the anagen phase transition and the composition for promoting FGF7 production.

The medicament can be provided in the form of an oral preparation or a topical preparation, and can be produced by formulation methods well known in the field of pharmaceutical preparations, such as the method described in the Japanese Pharmacopoeia, seventeenth Edition, General Rules for Preparation, [3] Monographs for Preparations, and the like.

The timing, frequency, and the duration of use of the medicament can be set in the same manner as the timing, frequency, and duration described above for the composition for maintaining the anagen phase, the composition for promoting the anagen phase transition, and the like.

The medicament can be used by those with thinning hair or hair loss such as androgenetic alopecia patients, female pattern alopecia patients, and the like, middle-aged and elderly persons at a risk of development or progression of thinning hair or hair loss, and the like.

Furthermore, either or both of the composition for promoting VEGF production and the composition for promoting FGF7 production provided in the form of oral compositions can be used by adding the same to various foods. The food to which either or both of the composition for promoting VEGF production and the composition for promoting FGF7 production is/are added is not particularly limited, and may be any as long as it is a food in the form generally served for meals or desserts. For example, either or both of the composition for promoting VEGF production and the composition for promoting FGF7 production is/are added to drinks, and a suitable flavor is added when desired, whereby a drink such as a beverage etc., can be provided. More specifically, either or both of the composition for promoting VEGF production and the composition for promoting FGF7 production can be added to, for example, juice, milk, confectionery, jelly, yogurt, candy, and the like.

Either or both of the composition for promoting VEGF production and the composition for promoting FGF7 production may be added to a food in an amount for ingestion per day such that the ingestion amount of one or more members selected from the group consisting of citrulline, tryptophan, and glutamine, or the ingestion amount of ornithine is the above-described daily ingestion amount determined for each of the composition for maintaining the anagen phase and the composition for promoting VEGF production, and the composition for promoting the anagen phase transition and the composition for promoting FGF7 production.

The present invention also provides a food for the prophylaxis or improvement of thinning hair or hair loss (hereinafter also to be referred to as “the food”).

The food contains either or both of the composition for promoting VEGF production and the composition for promoting FGF7 production provided as the above-described oral composition.

The food contains either or both of the composition for promoting VEGF production and the composition for promoting FGF7 production and, where necessary, food additives such as production agent, thickening stabilizer, gum base, emulsifier, preservative, antioxidant, gloss agent, pH adjuster, sweetener, bitter taste, acidulant, colorant, flavor, and the like. Alternatively, the food composition can be provided in various forms containing either or both of the composition for promoting VEGF production and the composition for promoting FGF7 production, and food or food starting materials, for example, drinks such as juice, beverage water, teas, and the like; milk products such as lactobacillus drinks, fermented milk, butter, cheese, yogurt, processing milk, defatted milk, and the like; meat products such as ham, sausage, hamburger steak, and the like; fish meat paste products such as boiled fish paste, tube-like fish sausage, satsuma-age, and the like; egg products such as rolled Japanese-style omelette, egg tofu, and the like; confectioneries such as cookie, jelly, chewing gum, candy, snack confectionery, frozen dessert, and the like; bread; noodles; pickles; smoked product; dried fish; food boiled down in soy; salt-preserved product; soups; seasonings, and may be provided as bottled food, canned food, retort pouch food. In addition, forms such as powder, granule, sheet, capsule, tablet, jelly, and the like can be provided.

The food can be ingested by those with thinning hair or hair loss such as androgenetic alopecia patients, female pattern alopecia patients, and the like, middle-aged and elderly persons at a risk of development or progression of thinning hair or hair loss, and the like.

Therefore, the food can also be provided as a food with health claims such as food for specified health uses for the prophylaxis or improvement of thinning hair or hair loss, a food with nutrient function claims, a food with functional claims attached with functional indications such as “promotion of hair growth”, “alleviation of fallen hair”, “reduction of fallen hair”, “hair thickening”, “alleviation of hair distress”, “suppression of scalp itchiness”, and the like, a food for special dietary uses such as food for middle-aged and elderly persons, and the like, health supplement, and the like.

Furthermore, either or both of the composition for promoting VEGF production and the composition for promoting FGF7 production provided as oral compositions can be used by adding to a food supplement.

The “food supplement” refers to one ingested to aid nutrition other than one ingested as a food, and also includes nutritional supplement, supplement, and the like. When either or both of the composition for promoting VEGF production and a composition for promoting FGF7 production is/are added to a food supplement, it/they can be prepared in a form such as tablet, capsule, powder, granule, suspension, chewable, syrup, and the like by adding other nutrition components and additives when desired.

The above-described food can be processed and produced by adding food additive as necessary to either or both of the composition for promoting VEGF production and the composition for promoting FGF7 production or adding either or both of the composition for promoting VEGF production and the composition for promoting FGF7 production to a food or food starting materials, and applying a general food production method.

The content of the composition for promoting VEGF production in the food can be appropriately determined according to the kind or form of the food, the level of thinning hair or hair loss-improving effect expected by the ingestion of the food, and the like. The total content of one or more members selected from the group consisting of citrulline, tryptophan, and glutamine (when contained in a salt form, calculated as an amount converted to a free form) is generally 0.000005 wt % to 99 wt %, preferably 0.00001 wt % to 95 wt %, more preferably 0.00005 wt % to 90 wt %, of the total weight of the food.

The content of the composition for promoting FGF7 production in the food in the content of ornithine (when contained in a salt form, as an amount converted to a free form) is generally 0.0001 wt % to 90 wt %, preferably 0.0005 wt % to 85 wt %, more preferably 0.001 wt % to 80 wt %.

From the aspects of the prophylactic or improving effect on thinning hair or hair loss, the food can preferably contain both the composition for promoting VEGF production and the composition for promoting FGF7 production.

The composition for promoting VEGF production and the composition for promoting FGF7 production are preferably contained in the food such that the content ratio of one or more members selected from the group consisting of citrulline, tryptophan, and glutamine, and ornithine (one or more members selected from the group consisting of citrulline, tryptophan, and glutamine:ornithine) is 1:0.1 to 1:0.5, more preferably 1:0.15 to 1:0.32.

The daily ingestion amount of the food can be set as an amount that can achieve ingestion of the aforementioned daily ingestion amount of one or more members selected from the group consisting of citrulline, tryptophan, and glutamine for the composition for maintaining the anagen phase and the composition for promoting VEGF production, or as an amount that can achieve ingestion of the aforementioned daily ingestion amount of ornithine for the composition for promoting the anagen phase transition and the composition for promoting FGF7 production.

Also provided herein is a commercial package containing either or both of the composition for promoting VEGF production and the composition for promoting FGF7 production, and written matter stating that either or both of the composition for promoting VEGF production and the composition for promoting FGF7 production can or should be used for the prophylaxis or improvement of thinning hair or hair loss.

Furthermore, also provided herein is a method for promoting VEGF or FGF7 production in a target animal in need of the promotion of VEGF or FGF7 production (hereinafter also to be referred to as “the method”).

The method as described herein includes use by ingestion, administration, or application of in a target animal in need of the promotion of VEGF or FGF7 production, one or more members selected from the group consisting of citrulline, tryptophan, and glutamine in an amount effective for promoting the VEGF production in the target animal, or ornithine in an amount effective for promoting the FGF7 production.

Alternatively, the method includes use of, in a target animal in need of the promotion of VEGF and FGF7 production, one or more members selected from the group consisting of citrulline, tryptophan, and glutamine, and ornithine, in an amount effective for promoting the VEGF and FGF7 production in the target animal.

As the target animal of the method, human and mammals other than human such as mouse, rat, hamster, rabbit, cat, dog, bovine, horse, donkey, swine, sheep, monkey, and the like can be encompassed.

In the case of human, the method can be applied to those with thinning hair or hair loss such as androgenetic alopecia patients, female pattern alopecia patients, and the like, middle-aged and elderly persons at a risk of development or progression of thinning hair or hair loss, and the like.

The amount of use of one or more members selected from the group consisting of citrulline, tryptophan, and glutamine, or the amount of use of ornithine in the method, is appropriately determined according to the kind, age, gender, body weight, the condition of thinning hair or hair loss, degree, and the like of the target animal.

Specifically, the composition for maintaining the anagen phase and the composition for promoting VEGF production can be used for a human or a target animal other than human in an amount similar to the above-described amount of use and at the frequency and duration described above.

In addition, the composition for promoting the anagen phase transition and the composition for promoting FGF7 production can be used for a human or a target animal other than human in an amount similar to the above-described amount of use and at the frequency and duration described above.

Furthermore, the use method of one or more members selected from the group consisting of citrulline, tryptophan, and glutamine, or ornithine in the method includes oral ingestion or oral administration, transdermal administration, and the like. These are preferred since they do not require the guidance and supervision of a doctor at a medical institution and can be performed conveniently.

### EXAMPLES

The present invention is explained in more detail in the following by way of Experimental Examples.

### Experimental Example 1: Study of Influence of Citrulline, Tryptophan, and Glutamine on VEGF Production in Hair Papilla Cells

Using a papilla cell growth medium (PCGM) (Toyobo Co., Ltd.), human hair papilla cells (Toyobo Co., Ltd.) were cultured for 4 days on a collagen-coated dish, and seeded on a collagen-coated 48 well plate at a cell density of 1.2×10⁴ cells/0.3 mL/well. The cells were cultured for one day in a carbon dioxide incubator (5 volume % carbon dioxide, 37° C.), and used for the evaluation of the VEGF production amount.

For the evaluation of the VEGF production amount, 20 kinds of amino acids were mixed at the composition shown in Table 1, the components other than amino acids were composed as in Dulbecco's modified Eagle medium (DMEM), glucose was added at 1 g/L, and the resulting medium was used as an evaluation medium.

TABLE 1

| amino acid | concentration (mg/L) |
|---|---|
| glycine | 7.50 |
| L-alanine | 8.90 |
| L-serine | 10.50 |
| L-threonine | 23.75 |
| L-cystine dihydrochloride | 15.75 |
| L-methionine | 7.50 |
| L-glutamine | 146.00 |
| L-asparagine | 13.20 |
| L-glutamic acid | 14.70 |
| L-aspartic acid | 13.30 |
| L-valine | 23.50 |
| L-leucine | 26.25 |
| L-isoleucine | 26.25 |
| L-phenylalanine | 16.50 |
| L-tyrosine disodium dihydrate | 26.00 |
| L-tryptophan | 4.00 |
| L-lysine hydrochloride | 36.50 |
| L-arginine hydrochloride | 21.00 |
| L-histidine hydrochloride monohydrate | 10.50 |
| L-proline | 11.50 |

L-citrulline, L-tryptophan, and L-glutamine were respectively dissolved in the evaluation medium to prepare each solution.

In the above-described hair papilla cell culture medium on the collagen-coated plate, the medium was replaced with the evaluation medium, the above-described respective solutions of L-citrulline, L-tryptophan, and L-glutamine were added at a given concentration, and the cells were cultured for 3 days at 37° C., 5 volume % carbon dioxide (n=4 for L-citrulline, L-tryptophan, and L-glutamine at each addition concentration).

Then, the supernatant of the culture medium was collected, centrifuged at 1500 rpm, and the supernatant was collected and cryopreserved at −30° C.

Similar culture and treatment were performed without adding each solution of L-citrulline, L-tryptophan, and L-glutamine and used as the control.

The concentration of VEGF in the supernatant of the culture was measured by thawing the collected supernatant and using the VEGF Enzyme-Linked ImmunoSorbent Assay (ELISA) kit (Abcam Plc.).

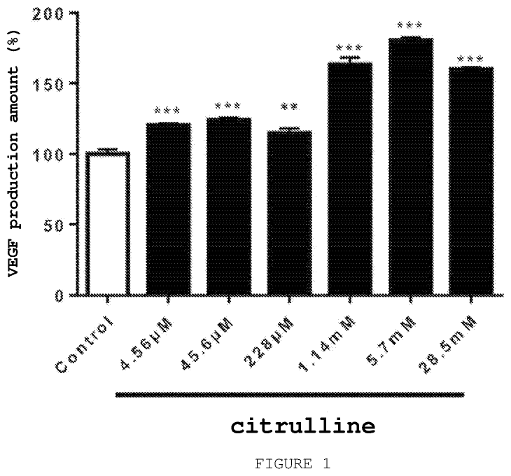

The measurement results were converted to relative values (%) with the VEGF concentration in the control as 100, and shown as mean±standard error in FIGS. 1 to 3.

As shown in FIGS. 1 to 3, a significant increase in the VEGF production amount was observed by adding each of L-citrulline, L-tryptophan, and L-glutamine as compared with the control.

In the case of L-citrulline, a significant increase in the VEGF production was observed by the addition at not less than 4.56 μM (FIG. 1, P<0.01 or P<0.001).

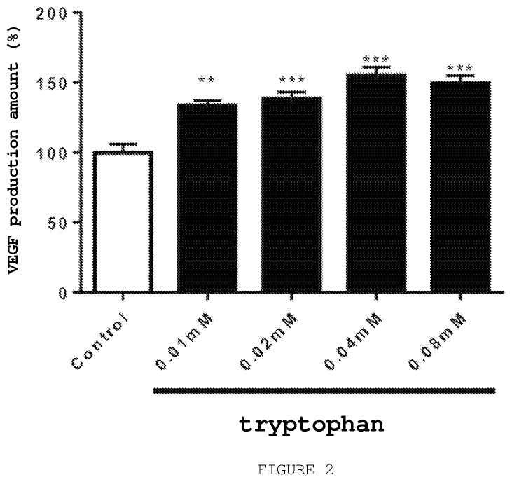

In the case of L-tryptophan, a significant increase in the VEGF production was observed by the addition at not less than 0.01 mM (10 μM) (FIG. 2, P<0.01 or P<0.001).

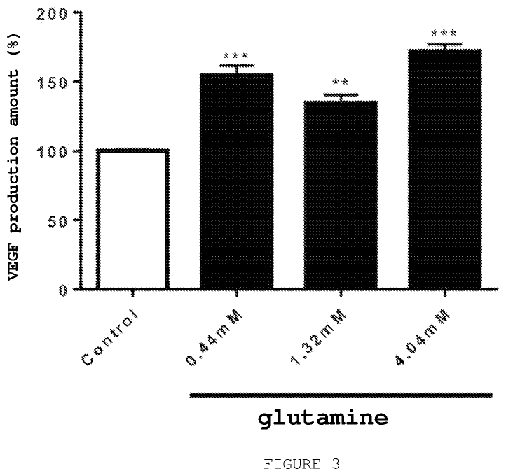

In the case of L-glutamine, a significant increase in the VEGF production was observed by the addition at not less than 0.44 mM (FIG. 3, P<0.01 or P<0.001).

It was confirmed from the above-described results of Experimental Example 1 that each of citrulline, tryptophan, and glutamine has an action on hair papilla cells to promote the production of VEGF which is a growth factor involved in the control of the hair cycle.

Therefore, the possibility was suggested that citrulline, tryptophan, and glutamine each may promote VEGF production by hair papilla cells, normalize the hair cycle by maintaining a normal anagen phase of the hair cycle, and promote hair growth.

### Experimental Example 2: Study of Influence of Ornithine on FGF7 Production in Hair Papilla Cells

In the same manner as in Experimental Example 1, human hair papilla cells were cultured for 4 days, and seeded on a collagen-coated 48 well plate at a cell density of 1.2×10⁴ cells/0.3 mL/well. The cells were cultured for one day in a carbon dioxide incubator (5 volume % carbon dioxide, 37° C.), and used for the evaluation of the FGF7 production amount. For the evaluation of the FGF7 production amount, the same evaluation medium as in Experimental Example 1 was used.

L-ornithine was dissolved in the evaluation medium to prepare a solution.

In the above-mentioned hair papilla cell culture medium on the collagen-coated plate, the medium was replaced with the evaluation medium, the above-mentioned solution of L-ornithine was added at a given concentration, and the cells were cultured for 3 days at 37° C., 5 volume % carbon dioxide (n=3 at each addition concentration).

Similar culture and treatment were performed without adding the solution of L-ornithine, and used as the control.

Then, the supernatant of the culture medium was collected, centrifuged at 1500 rpm, and the supernatant was collected and cryopreserved at −30° C.

The concentration of FGF7 in the supernatant of the culture was measured by thawing the collected supernatant and using the FGF7 Enzyme-Linked ImmunoSorbent Assay (ELISA) kit (Abcam Plc.).

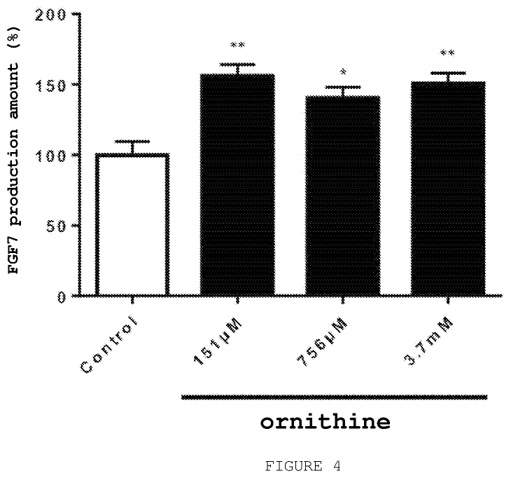

The measurement results were converted to relative values (%) with the FGF7 concentration in the control as 100, and shown as mean±standard error in FIG. 4.

As shown in FIG. 4, a significant increase in the FGF7 production amount was observed by adding L-ornithine at not less than 151 μM as compared with the control (P<0.05 or P<0.01).

It was confirmed from the above-described results of Experimental Example 2 that ornithine has an action on hair papilla cells to promote the production of FGF7 which is a growth factor involved in the control of the hair cycle.

Therefore, the possibility was suggested that ornithine may promote FGF7 production by hair papilla cells, normalize the hair cycle by promoting transition from the telogen phase to the anagen phase of the hair cycle, and promote new hair growth.

### Experimental Example 3: Study of Influence of Citrulline on Proliferation of Hair Papilla Cells

In the same manner as in Experimental Example 1, human hair papilla cells were cultured for 4 days, and seeded on a collagen-coated 96 well plate at a cell density of 2.5×10³ cells/0.1 mL/well. The cells were cultured for one day in a carbon dioxide incubator (5 volume % carbon dioxide, 37° C.), and used for the evaluation of the proliferation of hair papilla cells. For the evaluation of the proliferation of hair papilla cells, the same evaluation medium as used in Experimental Example 1 was used.

L-citrulline was dissolved in the evaluation medium to prepare a solution.

In the above-mentioned hair papilla cell culture medium on the collagen-coated plate, the medium was replaced with the evaluation medium, the above-mentioned solution of L-citrulline was added to a concentration of 2 mM, and the cells were cultured for one day at 37° C., 5 volume % carbon dioxide (n=7).

Then the Cell Counting kit-8 (DOJINDO LABORATORIES) was added to each well by 10 μL, the plate was placed in a carbon dioxide incubator (5 volume % carbon dioxide, 37° C.) for 2 hr, and the absorbance at 450 nm was measured using a microplate reader.

Similar culture and treatment were performed without adding the solution of L-citrulline and used as the control.

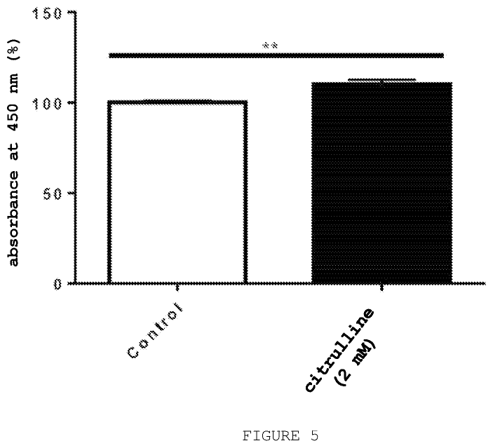

The measurement results were converted to relative values (%) with the absorbance in the control as 100, and shown as mean±standard error in FIG. 5.

As shown in FIG. 5, a significant increase in the number of hair papilla cells was observed by adding L-citrulline at 2 mM as compared with the control (P<0.01).

It was confirmed from the above-described results of Experimental Example 3 that citrulline promotes the proliferation of hair papilla cells.

Therefore, the possibility was suggested that citrulline may promote the proliferation of hair papilla cells which regulate hair growth, activate hair matrix cells, promote hair thickening, and grow long and thick hair, by increasing VEGF secretion.

### Experimental Example 4: Study of Influence of Ornithine on Proliferation of Hair Papilla Cells

In the same manner as in Experimental Example 1, human hair papilla cells were cultured for 4 days, and seeded on a collagen-coated 96 well plate at a cell density of 2.5×10³ cells/0.1 mL/well. The cells were cultured for one day in a carbon dioxide incubator (5 volume % carbon dioxide, 37° C.), and used for the evaluation of the proliferation of hair papilla cells. For the evaluation of the proliferation of hair papilla cells, a medium having the same amino acid composition as the evaluation medium used in Experimental Example 1 and other components diluted at a ¼ concentration was used as the evaluation medium.

L-ornithine was dissolved in the above-mentioned evaluation medium to prepare a solution.

In the above-mentioned hair papilla cell culture medium on the collagen-coated plate, the medium was replaced with the evaluation medium, the above-mentioned solution of L-ornithine was added to a given concentration, and the cells were cultured for one day at 37° C., 5 volume % carbon dioxide (n=6 at each concentration).

Then the Cell Counting kit-8 (DOJINDO LABORATORIES) was added to each well by 10 μL, the plate was placed in a carbon dioxide incubator (5 volume % carbon dioxide, 37° C.) for 2 hr, and the absorbance at 450 nm was measured using a microplate reader.

Similar culture and treatment were performed without adding the solution of L-ornithine and used as the control.

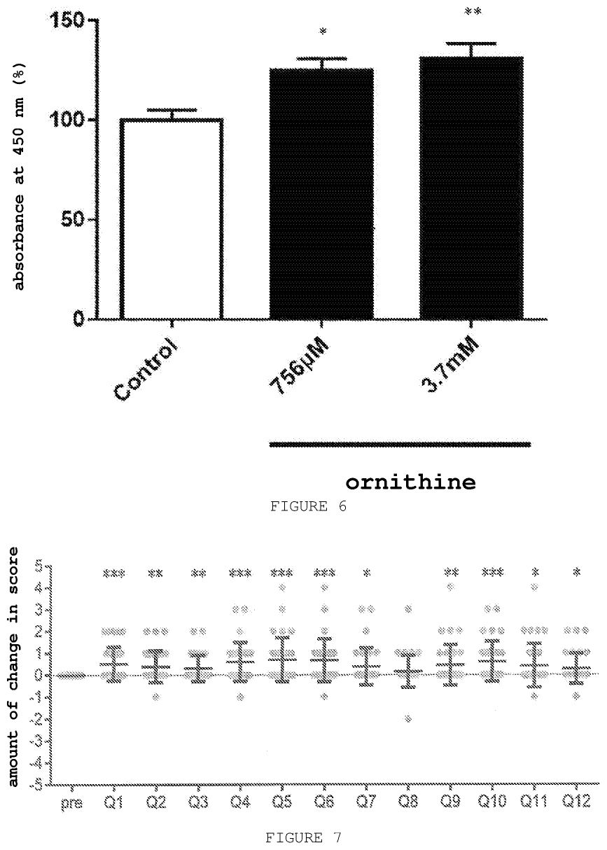

The measurement results were converted to relative values (%) with the absorbance in the control as 100, and shown as mean±standard error in FIG. 6.

As shown in FIG. 6, a significant increase in the number of hair papilla cells was observed by adding L-ornithine at 756 μM or 3.7 mM as compared with the control (P<0.05 or P<0.01).

It was confirmed from the above-mentioned results of Experimental Example 4 that ornithine promotes the proliferation of hair papilla cells.

Therefore, the possibility was suggested that ornithine may promote the proliferation of hair papilla cells which regulate hair growth, promote hair thickening, and grow long and thick hair, in addition to increase of FGF7 secretion.

### Experimental Example 5: Use Test of Citrulline and Ornithine in Human

As in the following, use test of citrulline and ornithine in human was conducted as described below.

(1) Test Subject

The Subjects of the Test were Healthy Males Aged 20 to 59 and females aged 40 to 59 who had concerns about hair. Those who were taking citrulline, those who were taking ornithine, those who were outpatient to the hospital or taking medication for the treatment of diseases including hair-related diseases, those who were pregnant or breastfeeding, and those who had a plan to be pregnant or give birth were excluded from the test subjects.

(2) Test Method

“Kyowa Hakko Bio Citrulline” (220 mg per granule, citrulline content=800 mg/6 granules) and “Kyowa Hakko Bio Ornithine” (250 mg per granule, ornithine content=800 mg/6 granules) (Kyowa Hakko Bio Co., Ltd.) as the test foods were orally ingested by the test subjects for 12 weeks (6 granules each, once a day, every day between after dinner and before going to bed).

The test design was a single-group open test, and the test subjects compared and evaluated the conditions before and after ingestion of the test food. The aforementioned evaluation was conducted by answering the questionnaire shown in Table 2.

As shown in Table 2, the questionnaire had hair-related evaluation items (Q1 to Q12) as the main evaluation items, and 11 point scores of 0 to 10 were set for all evaluation items. The score before ingestion of the test food was set to 5, and after ingestion of the test food, the test subjects subjectively selected and reported the score at a frequency of once every two weeks.

TABLE 2

| question No. | score setting (condition before test food ingestion is 5) | | | | | | | | | | |
|---|---|---|---|---|---|---|---|---|---|---|---|
| and item | 0 | 1 | 2 | 3 | 4 | 5 | 6 | 7 | 8 | 9 | 10 |

| | | | | | |
|---|---|---|---|---|---|
| primary | Q1 | whole hair | reduced | ----------------- | increased |
| evaluation | Q2 | degree of | worried | ----------------- | not worried |
| item | | worry of | | | |
| | | thinning hair | | | |
| | Q3 | amount of | reduced | ----------------- | increased |
| | | newly grown | | | |
| | | hair | | | |
| | Q4 | amount of | increased | ----------------- | reduced |
| | | fallen hair | | | |
| | Q5 | amount of | increased | ----------------- | reduced |
| | | hair adhered | | | |
| | | to the pillow | | | |
| | | when waking | | | |
| | | up | | | |
| | Q6 | amount of | increased | ----------------- | reduced |
| | | hair fallen | | | |
| | | out during | | | |
| | | hair washing | | | |
| | Q7 | hair length | decelerated | ----------------- | accelerated |
| | | growth | | | |
| | Q8 | amount of | increased | ----------------- | reduced |
| | | gray hair | | | |
| | Q9 | thickness of | thinned | ----------------- | thickened |
| | | hair | | | |
| | Q10 | volume of | reduced | ----------------- | increased |
| | | hair | | | |
| | Q11 | itchiness of | itching | ----------------- | no itching |
| | | scalp | | | |
| | Q12 | conspicuous | conspicuous | ----------------- | not |
| | | head skin | | | conspicuous |
| secondary | Q13 | skin dryness | dry | ----------------- | not dry |
| evaluation | Q14 | skin shine | shiny | ----------------- | not shiny |
| item | Q15 | feeling of | remain | ----------------- | not remain |
| | | fatigue when | | | |
| | | waking up | | | |
| | Q16 | sleep | unable to | ----------------- | can sleep |
| | | condition | sleep | | |
| | Q17 | cold hands | cold | ----------------- | not cold |
| | | and feet | | | |

(3) Analysis of Evaluation Results

The population (PPS), excluding those whose ingestion rate of the test food was 50% or less and those who did not respond to the questionnaire, was the subject of data analysis. Analysis was performed using the analysis soft “Microsoft Excel 2016, GraphPad Prism 6”.

Regarding the response results of the questionnaire, the score before test food ingestion was subtracted from the score after 2, 4, 6, 8, 10, and 12 weeks after the test food ingestion, the amount of change over time was calculated, and comparison with that before test food ingestion was performed using the Kruskal-Wallis test. In addition, with regard to the amount of change obtained by subtracting the score before test food ingestion from the score 12 weeks after the test food ingestion, comparison between before and after test food ingestion was performed using the Wilcoxon signed rank test. The significance level was less than 5% in a two-tailed test, and a trend was set at between not less than 5% and less than 10%.

(4) Evaluation Results

Of the test subjects whose ingestion rate of the test food exceeded 50%, the above-mentioned analysis was performed on 31 subjects who responded to the questionnaire. No subject discontinued the test due to adverse events or exacerbation of health conditions.

For each evaluation item relating to hair, the amount of change in score over time (the amount of change from before test food ingestion, the amount of change in score before test food ingestion is set to 0) is shown in Table 3.

In addition, the amount of change in score at the end of test food ingestion was calculated and shown in FIG. 7 as mean±standard deviation. If no response was obtained after 12 weeks, which corresponds to after the test food ingestion, the amount of change in score was calculated using the response results at after 10 weeks.

TABLE 3

| | | | whole | number of | |
|---|---|---|---|---|---|
| | | | (n = 31) | response | p-value |
| Q1 | 2 | weeks | 0.0 ± 0.2 | 30 | NS |
| (whole hair) | 4 | weeks | 0.2 ± 0.8 | 31 | NS |
| | 6 | weeks | 0.3 ± 0.6 | 29 | NS |
| | 8 | weeks | 0.4 ± 0.6 | 28 | p < 0.05 |
| | 10 | weeks | 0.5 ± 0.8 | 29 | p < 0.01 |
| | 12 | weeks | 0.5 ± 0.7 | 25 | p < 0.05 |
| Q2 | 2 | weeks | 0.0 ± 0.3 | 31 | NS |
| (degree of worry | 4 | weeks | 0.2 ± 0.8 | 31 | NS |
| of thinning hair) | 6 | weeks | 0.3 ± 0.5 | 31 | NS |
| | 8 | weeks | 0.3 ± 0.5 | 28 | p < 0.1 |
| | 10 | weeks | 0.4 ± 0.8 | 28 | p < 0.01 |
| | 12 | weeks | 0.3 ± 0.7 | 24 | NS |
| Q3 | 2 | weeks | 0.0 ± 0.2 | 30 | NS |
| (amount of newly | 4 | weeks | 0.2 ± 0.4 | 31 | NS |
| grown hair) | 6 | weeks | 0.2 ± 0.5 | 30 | NS |
| | 8 | weeks | 0.2 ± 0.4 | 28 | NS |
| | 10 | weeks | 0.3 ± 0.6 | 29 | p < 0.01 |
| | 12 | weeks | 0.3 ± 0.6 | 24 | p < 0.1 |
| Q4 | 2 | weeks | 0.0 ± 0.5 | 31 | NS |
| (amount of fallen | 4 | weeks | 0.1 ± 0.8 | 31 | NS |
| hair) | 6 | weeks | 0.4 ± 0.7 | 30 | p < 0.05 |
| | 8 | weeks | 0.4 ± 0.8 | 28 | NS |
| | 10 | weeks | 0.6 ± 0.9 | 29 | p < 0.05 |
| | 12 | weeks | 0.5 ± 0.8 | 23 | p < 0.05 |
| Q5 | 2 | weeks | 0.1 ± 0.3 | 31 | NS |
| (amount of hair | 4 | weeks | 0.2 ± 0.6 | 31 | NS |
| adhered to pillow | 6 | weeks | 0.3 ± 0.5 | 31 | p < 0.1 |
| when waking up) | 8 | weeks | 0.4 ± 0.7 | 28 | p < 0.1 |
| | 10 | weeks | 0.6 ± 1.1 | 29 | p < 0.05 |
| | 12 | weeks | 0.7 ± 0.9 | 24 | p < 0.001 |
| Q6 | 2 | weeks | 0.0 ± 0.5 | 31 | NS |
| (amount of hair | 4 | weeks | 0.3 ± 0.9 | 31 | p < 0.1 |
| fallen out during | 6 | weeks | 0.3 ± 0.8 | 31 | NS |
| hair washing) | 8 | weeks | 0.3 ± 0.8 | 28 | NS |
| | 10 | weeks | 0.7 ± 1.0 | 29 | p < 0.01 |
| | 12 | weeks | 0.5 ± 0.9 | 22 | p < 0.05 |
| Q7 | 2 | weeks | 0.0 ± 0.2 | 31 | NS |
| (hair length | 4 | weeks | 0.1 ± 0.3 | 31 | NS |
| growth) | 6 | weeks | 0.2 ± 0.4 | 30 | NS |
| | 8 | weeks | 0.1 ± 0.4 | 27 | NS |
| | 10 | weeks | 0.3 ± 0.6 | 29 | p < 0.1 |
| | 12 | weeks | 0.3 ± 0.9 | 24 | NS |
| Q8 | 2 | weeks | 0.0 ± 0.5 | 31 | NS |
| (amount of gray | 4 | weeks | 0.2 ± 0.7 | 31 | NS |
| hair) | 6 | weeks | 0.1 ± 0.6 | 31 | NS |
| | 8 | weeks | 0.0 ± 0.4 | 28 | NS |
| | 10 | weeks | 0.1 ± 0.6 | 29 | NS |
| | 12 | weeks | 0.0 ± 0.6 | 24 | NS |
| Q9 | 2 | weeks | 0.1 ± 0.2 | 31 | NS |
| (thickness of | 4 | weeks | 0.2 ± 0.6 | 31 | NS |
| hair) | 6 | weeks | 0.2 ± 0.5 | 31 | NS |
| | 8 | weeks | 0.1 ± 0.4 | 28 | NS |
| | 10 | weeks | 0.4 ± 0.9 | 29 | p < 0.05 |
| | 12 | weeks | 0.3 ± 0.6 | 23 | p < 0.05 |
| Q10 | 2 | weeks | 0.2 ± 0.5 | 31 | NS |
| (volume of hair) | 4 | weeks | 0.3 ± 0.8 | 31 | NS |
| | 6 | weeks | 0.2 ± 0.4 | 31 | NS |
| | 8 | weeks | 0.3 ± 0.5 | 27 | NS |
| | 10 | weeks | 0.6 ± 0.9 | 29 | p < 0.01 |
| | 12 | weeks | 0.5 ± 0.8 | 24 | p < 0.01 |
| Q11 | 2 | weeks | 0.1 ± 0.5 | 31 | NS |
| (itchiness of | 4 | weeks | 0.2 ± 0.6 | 31 | NS |
| scalp) | 6 | weeks | 0.1 ± 0.8 | 31 | NS |
| | 8 | weeks | 0.2 ± 0.7 | 28 | NS |
| | 10 | weeks | 0.3 ± 0.7 | 29 | NS |
| | 12 | weeks | 0.5 ± 1.1 | 24 | NS |
| Q12 | 2 | weeks | 0.0 ± 0.2 | 31 | NS |
| (conspicuous head | 4 | weeks | 0.0 ± 0.7 | 30 | NS |
| skin) | 6 | weeks | 0.1 ± 0.5 | 31 | NS |
| | 8 | weeks | 0.1 ± 0.3 | 27 | NS |
| | 10 | weeks | 0.3 ± 0.7 | 29 | p < 0.05 |
| | 12 | weeks | 0.3 ± 0.7 | 24 | NS |

As shown in Table 3, it was observed that the amount of change in score for Q1 (amount of hair) increased significantly on and after 8 weeks from the test food ingestion, and the number of test subjects who felt that the amount of hair increased due to the test food ingestion increased.

It was observed that the amount of change in the score for Q2 (degree of worry of thinning hair) tended to increase after 8 weeks, a significant increase was observed after 10 weeks, and the number of test subjects who did not worry about thinning hair increased.

It was observed that the amount of change in score for Q3 (amount of newly grown hair) showed a significant increase after 10 weeks, tended to increase after 12 weeks, and the number of test subjects increased who felt that the amount of newly grown hair increased.

It was observed that the amount of change in score for Q4 (amount of fallen hair) increased significantly on and after 6 weeks and 10 weeks, and the number of test subjects increased who felt that the amount of fallen hair decreased.

It was observed that the scores for Q5 (amount of hair adhered to the pillow when waking up), Q6 (amount of hair fallen out during hair washing), Q9 (thickness of hair), and Q10 (volume of hair) each significantly increased on and after 10 weeks, and the test subjects who felt that fallen hair decreased and the test subjects who felt that the hair thickened increased.

It was shown that the amount of change in score for Q12 (conspicuous head skin) increased significantly 10 weeks after the test food ingestion, and the number of test subjects increased who felt that the skin became less conspicuous.

The amount of change in score for Q7 (hair length growth) tended to increase only after 10 weeks.

No significant change was observed in the amount of change in scores for Q8 (amount of gray hair) and Q11 (itchiness of scalp). However, positive values were obtained, and the presence of test subjects was observed who felt that gray hair and itchiness of the scalp decreased.

As shown in FIG. 7, the amount of change in the score significantly increased in the evaluation items other than Q8 (significant at P<0.001 for Q1, Q4 to Q6, and Q10, significant at P<0.01 for Q2, Q3, and Q9, significant at P<0.05 for Q7, Q11, and Q12) as compared with before the test food ingestion (pre), and the usefulness of test food ingestion for worry of hair was suggested.

From the above-described results of Experimental Example 5, it was suggested that oral ingestion of citrulline and ornithine makes hair grow thicker and is effective in improving thinning hair and fallen hair.

### Experimental Example 6: Study of Influence of Content Ratio of Citrulline and Ornithine

In an animal experiment using male mice, the influence of the content ratio of citrulline and ornithine during oral administration on the plasma concentration of citrulline was studied as follows.

(1) Experimental Method

(i) 5-Week-old male C3H/HeN mice (CLEA Japan, Inc.) were received and raised in an animal breeding room under Specific Pathogen Free (SPF). After providing an acclimation period, the mice were divided into groups at the time of 7 weeks of age and subjected to the test. The mice were reared in polycages (small) with paper bedding (pepper clean), one per cage, and freely given solid feed CRF1 (Oriental Yeast Co., Ltd.) and tap water. The breeding room was set to 12 hours light and 12 hours dark (lighting: 7:00 to 19:00), temperature 23±1° C., and humidity 55±10%.

(ii) Preparation of Test Solution

As the test solutions, L-citrulline aqueous solution (0.2 g/mL) and L-ornithine hydrochloride aqueous solution (0.2 g/mL) were prepared using L-citrulline (Nacalai Tesque, Inc.: Cat No. 09113-31), L-ornithine hydrochloride (Nacalai Tesque, Inc., Ltd.: Cat No. 25718-92), and water for injection (Otsuka Pharmaceutical Factory, Inc.), and a mixed solution of citrulline and ornithine hydrochloride was prepared as shown in Table 4, and they were used as test solutions.

TABLE 4

| | citrulline | ornithine | distilled |
|---|---|---|---|
| L-citrulline:L-ornithine | solution | solution | water |
| hydrochloride weight | amount | amount | amount |
| ratio (molar ratio) | (mL) | (mL) | (mL) |
| 1:0(1:0) | 0.5 | 0 | 0.5 |
| 1:0.15(1:0.16) | 0.5 | 0.075 | 0.425 |
| 1:0.3(1:0.31) | 0.5 | 0.15 | 0.35 |
| 1:0.6(1:0.62) | 0.5 | 0.3 | 0.2 |
| 1:1(1:1.04) | 0.5 | 0.5 | 0 |

(iii) Administration of Test Solution and Measurement of Plasma Concentration

The mice were divided into 6 groups (10 to 15 mice/group) and, with the day on which the grouping was performed as day 1, each test solution was orally administered to each group at 10 mL/kg once a day for 5 days from day 1 to day 5. As a control, the same amount of distilled water was administered. The heart blood was collected under isoflurane anesthesia at 30 min, 1 hr, and 2 hr after administration on day 5, and the amino acid concentration of the separated plasma was measured using UF-Amino Station (Shimadzu Corporation).

(IV) Statistical Analysis of Measurement Results

The statistical analysis of the measurement results was performed using GraphPad Prism 6 (GraphPad Software, Inc.). Significant digits after the decimal point were 2 digits, and a significant difference test of blood concentration after elapse of each time was performed at p<0.05 using the unpaired Tukey's multiple comparison test, for each group except the distilled water administration group (DW).

(2) Results

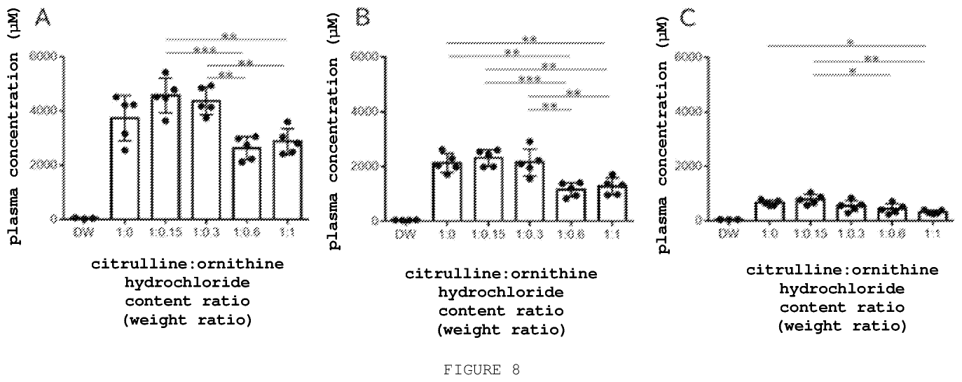

FIG. 8 shows the measurement results of the plasma concentration of citrulline at 30 min, 1 hr, and 2 hr after administration as mean±standard deviation.

As shown in FIG. 8, it was observed that the citrulline plasma concentration 30 min after administration was significantly high in a test solution (molar ratio=1:0.16) administration group in which the content ratio of citrulline and ornithine hydrochloride (citrulline:ornithine hydrochloride) was 1:0.15 (weight ratio), and a test solution (molar ratio=1:0.31) administration group in which the content ratio was 1:0.3 (weight ratio), as compared with a test solution (molar ratio=1:0.62) administration group in which the content ratio was 1:0.6 (weight ratio), and a test solution (molar ratio=1:1.04) administration group in which the content ratio was 1:1 (weight ratio) (A).

It was observed that the citrulline plasma concentration 1 hr after administration was significantly high in a test solution (molar ratio=1:0) administration group in which the content ratio of citrulline and ornithine hydrochloride (citrulline:ornithine hydrochloride) was 1:0 (weight ratio), a test solution (molar ratio=1:0.16) administration group in which the content ratio was 1:0.15 (weight ratio), and a test solution (molar ratio=1:0.31) administration group in which the content ratio was 1:0.3 (weight ratio), as compared with a test solution (molar ratio=1:0.62) administration group in which the content ratio was 1:0.6 (weight ratio), and a test solution (molar ratio=1:1.04) administration group in which the content ratio was 1:1 (weight ratio) (B).

It was observed that the citrulline plasma concentration 2 hr after administration was significantly high in a test solution (molar ratio=1:0) administration group in which the content ratio of citrulline and ornithine hydrochloride (citrulline:ornithine hydrochloride) was 1:0 (weight ratio), and a test solution (molar ratio=1:0.16) administration group in which the content ratio was 1:0.15 (weight ratio), as compared with a test solution (molar ratio=1:1.04) administration group in which the content ratio was 1:1 (weight ratio), and also significantly high in a test solution (molar ratio=1:0.16) administration group in which the content ratio was 1:0.15 (weight ratio) as compared with a test solution (molar ratio=1:0.62) administration group in which the content ratio was 1:0.6 (weight ratio) (C).

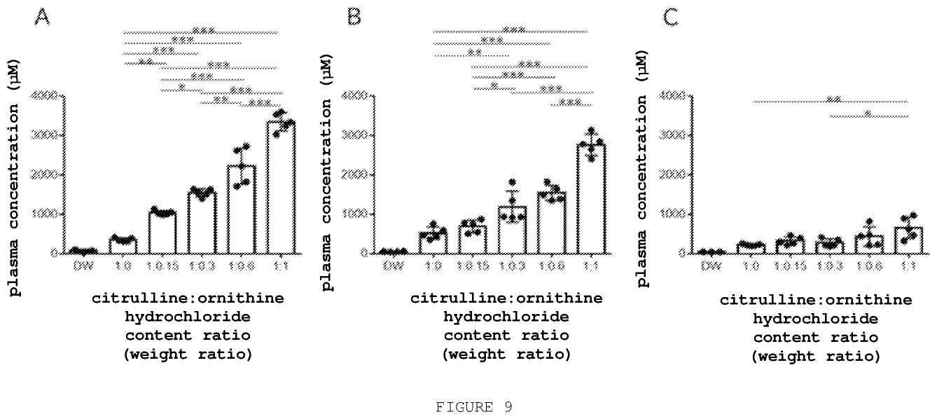

In addition, FIG. 9 shows the measurement results of the plasma concentration of ornithine at 30 min, 1 hr, and 2 hr after administration as mean±standard deviation.

As shown in FIG. 9, it was observed that the plasma concentration of ornithine at 30 min and 1 hr after administration was significantly high in an ornithine content ratio dependent manner (A, B).

It was observed that the ornithine plasma concentration 2 hr after administration was significantly high in a test solution (molar ratio=1:1.04) administration group in which the content ratio of citrulline and ornithine hydrochloride (citrulline:ornithine hydrochloride) was 1:1 (weight ratio) as compared with a test solution (molar ratio=1:0) administration group in which the content ratio was 1:0 (weight ratio), and a test solution (molar ratio=1:0.31) administration group in which the content ratio was 1:0.3 (weight ratio).

From the above-described results of Experimental Example 6, it was suggested that citrulline absorption is effectively improved, and hair thickening promoting effect and hair cycle normalization promoting effect, and further, thinning hair or hair loss preventing or improving effect are further improved, when the content ratio of citrulline and ornithine (citrulline:ornithine) is 1:0.16-1:0.31 in molar ratio.

### INDUSTRIAL APPLICABILITY

As described in detail above, a composition for promoting hair thickening can be provided.

The composition for promoting hair thickening can promote hair thickening by promoting VEGF or FGF7 production in hair papilla cells and proliferation of hair papilla cells, and grow long, thick, and healthy hair.

Moreover, a composition for maintaining the anagen phase of the hair cycle and a composition for promoting VEGF production can be provided.

The composition for maintaining the anagen phase of the hair cycle can promote division and proliferation of hair matrix cells, suppress shortening of the anagen phase of hair, and normalize the hair cycle by maintaining the normal anagen phase, by promoting VEGF production in hair papilla cells.

The composition for promoting VEGF production can promote VEGF production in hair papilla cells, promote division and proliferation of hair matrix cells, suppress shortening of the anagen phase of hair, normalize the hair cycle by maintaining the normal anagen phase, and promote thickening of hair by promoting proliferation of hair papilla cells.

Furthermore, a composition for promoting transition from the telogen phase to the anagen phase of the hair cycle and a composition for promoting FGF7 production can be provided.

The composition for promoting transition from the telogen phase to the anagen phase of the hair cycle can shorten the telogen phase which is from the degeneration of the hair root until a hair begins to grow anew, normalize the hair cycle by promoting the transition from the telogen phase to the anagen phase, promote the new hair growth by promoting division and proliferation of hair matrix cells, by promoting FGF7 production in hair papilla cells.

The composition for promoting FGF7 production can shorten the telogen phase which is from the degeneration of the hair root until a hair begins to grow anew by promoting FGF7 production in dermal papilla cells, normalize the hair cycle by promoting the transition from the telogen phase to the anagen phase, and promote the new hair growth by promoting the division and proliferation of hair matrix cells.

The composition for promoting hair thickening, the composition for maintaining the anagen phase of the hair cycle, and the composition for promoting VEGF production can be preferably used as hair-growing agents. The composition for promoting transition from the telogen phase to the anagen phase of the hair cycle and the composition for promoting FGF7 production can be preferably used as new-hair-growing agents.

Furthermore, a composition for promoting normalization of the hair cycle can be provided.

The composition for promoting the normalization of the hair cycle can normalize the hair cycle by either or both of the action of maintaining the anagen phase of the hair cycle by promoting VEGF production, and the action of promoting transition from the telogen phase to the anagen phase of the hair cycle by promoting FGF7 production.

The composition for promoting hair thickening, the composition for maintaining the anagen phase of the hair cycle, and the composition for promoting VEGF production, the composition for promoting transition from the telogen phase to the anagen phase of the hair cycle and the composition for promoting FGF7 production, and the composition for promoting normalization of the hair cycle can favorably improve or prevent thinning hair or hair loss, and are particularly useful for androgenetic alopecia and female pattern alopecia.

1. A method for promoting hair thickening or normalizing the hair cycle in a subject in need thereof, comprising administering or applying to the subject an effective amount of a composition comprising one or more members selected from the group consisting of citrulline, tryptophan, glutamine, and ornithine, or an effective amount of a composition comprising one or more members selected from the group consisting of citrulline, tryptophan, and glutamine, and ornithine.

2. The method according to claim 1, wherein the effective amount is 0.1 μM to 1 M as the total content of one or more members selected from the group consisting of citrulline, tryptophan, glutamine, and ornithine in the composition.

3. The method according to claim 1, wherein a content ratio of the one or more members selected from the group consisting of citrulline, tryptophan, and glutamine, and ornithine (one or more members selected from the group consisting of citrulline, tryptophan, and glutamine:ornithine) in the composition is 1:0.1 to 1:0.5 in molar ratio.

4. A method for maintaining the anagen phase of the hair cycle or promoting VEGF production in a subject in need thereof, comprising administering or applying to the subject an effective amount of a composition comprising one or more members selected from the group consisting of citrulline, tryptophan, and glutamine.

5. The method according to claim 4, wherein the effective amount is 0.1 μM to 1 M as the total content of one or more members selected from the group consisting of citrulline, tryptophan, and glutamine in the composition.

6. A method for promoting transition from the telogen phase to the anagen phase of the hair cycle or promoting FGF7 production in a subject in need thereof, comprising administering or applying to the subject an effective amount of a composition comprising ornithine.

7. The method according to claim 6, wherein the effective amount is 1 μM to 1 M as the content of ornithine in the composition.

8. A method for preventing or improving thinning hair or hair loss in a subject in need thereof, comprising administering or applying to the subject an effective amount of a composition comprising:
i) one or more members selected from the group consisting of citrulline, tryptophan, and glutamine; or
ii) ornithine; or
iii) one or more members selected from the group consisting of citrulline, tryptophan, and glutamine, and ornithine.

9. The method according to claim 8, wherein the composition is applied topically.

10. The method according to claim 9, wherein the composition is topically applied to skin.

11. The method according to claim 9, wherein the composition is a hair cosmetic or a scalp cosmetic.

12. A method for suppressing or ameliorating scalp itchiness in a subject in need thereof, comprising administering or applying to the subject an effective amount of a composition comprising:
i) one or more members selected from the group consisting of citrulline, tryptophan, and glutamine; or
ii) ornithine; or
iii) one or more members selected from the group consisting of citrulline, tryptophan, and glutamine, and ornithine.
